(19) United States
(12) Patent Application Publication
(10) Pub. No.: US 2003/0176748 A1
(43) Pub. Date: Sep. 18, 2003

(54) Direct condensation

(76) Inventors: Peter Kammerhofer, Burgkirchen (DE); Ingolf Mielke, Burgkirchen (DE); Horst Ertl, Pleiskirchen (DE); Dieter Jaculi, Burgkirchen (DE); Manfred Stoeger, Burgkirchen (DE)

Correspondence Address: Ronald R Santucci, Frommer Lawrence & Haug, 745 Fifth Avenue, New York, NY, 10151, US

(21) Appl. No.: 10/240,582

(22) PCT Filed: Feb. 13, 2002
(86) PCT No.: PCT/EP02/01499

(30) Foreign Application Priority Data
Feb. 13, 2001 (DE) 101 07 091.8

(51) Int. Cl. C07C 17/156

(52) U.S. Cl. 570/203

(57) ABSTRACT

A process is described for the preparation of 1,2-dichloroethane by reacting ethene with hydrogen chloride and an oxygen-containing gas in an oxychlorination reactor with formation of a reaction gas, wherein the reaction gas is, after filtration, condensed without prior quenching.

The present invention relates to a process for the preparation of 1,2-dichloroethane (EDC) by reacting ethene with hydrogen chloride and an oxygen-containing gas in an oxychlorination reactor, wherein a reaction gas is formed.

Oxychlorination is understood to be the reaction of an alkene—in this instance ethene—with hydrogen chloride and oxygen or an oxygen-containing gas such as air to form a saturated chlorinated alkane—in this instance 1,2-dichloroethane, also referred to hereinafter as “EDC”—, the reaction taking place according to the equation

C₂H₄+2HCl +½O₂→Cl—CH₂—CH₂—CI+H₂O.

The subsidiary reaction product (water) of that reaction can, however, together with unreacted starting material (hydrogen chloride), form hydrochloric acid, which is very highly corrosive, so that when carrying out such a process appropriately resistant—and, consequently, expensive—materials have to be used for the apparatus in order to carry out the process.

On a large industrial scale, that process is frequently carried out in a fluidised bed, the catalyst consisting, for example, substantially of copper chloride on an aluminium oxide carrier.

From German Offenlegungsschrift DE 41 32 030 there is known a process for the removal of catalyst fragments, which are passed out from the reaction zone together with the crude EDC gas stream. In that process, the catalyst fragments are separated from the crude EDC gas stream in a cleaning zone operated under dry conditions. Preferred embodiments of that process are distinguished by the fact that the catalyst fragments are separated out at a dust separator or in an electrofilter as the cleaning zone, it being possible for the dust separator to be equipped with bag filters which are cleaned with compressed recycle gas. After separating out the catalyst fragments, the gas stream is cooled with water and condensed, that is to say quenched.

It is, furthermore, possible for the catalyst fragments separated out in the cleaning zone to be freed from adsorbed reaction products in a desorption zone located downstream. The desorption zone can be operated at a temperature of from 50 to 350° C., preferably from 150 to 180° C., by means of gasifying, or at reduced pressure; and, for the purpose of gasifying, air, nitrogen or recycle gas (gas that is circulated in a loop for fluidisation of the catalyst) can be used and the catalyst fragments can be treated in the desorption zone for from 0.5 to 5 hours, preferably from 1 to 2 hours, at elevated temperature.

Such a process avoids the formation of waste water contaminated with heavy metal and inorganic slurry, when the water that is formed and the washing water that is used in working-up are removed.

DE 195 46 068 A1 relates to a process for reducing the catalyst usage and contaminated catalyst waste in the preparation of EDC according to the oxychlorination process. In that process, the catalyst fragments are separated out from the crude EDC gas stream in a separating zone operated under dry conditions. The catalyst fragments are classified and certain particle size fractions are returned to the reaction zone. In that process too, after the catalyst fragments have been separated out, the gas stream is cooled with water and condensed.

DE-A-197 53 165 discloses a process for the preparation of EDC by oxychlorination, wherein the reaction gas is freed from catalyst in the reactor by means of very fine filtration and so is retained in the reactor. The reaction gas freed from catalyst is then passed into a quenching column and condensed in known manner.

It is furthermore known from the prior art (Ullmann's Encyclopedia of Industrial Chemistry, Vol. A6, 1986, p. 269) for the hot reaction gases from a fluidised-bed reactor, which gases also comprise unreacted HCI gas in addition to EDC and water, to be quenched using an aqueous solution without further treatment. In that method, catalyst fragments that have not been separated out and unreacted hydrogen chloride from the oxychlorination of ethene are washed out. It is possible to use, as washing liquid, both external water and water that is formed during the reaction, the so-called water of reaction. EDC is, together with water from the quench, distilled off and condensed.

All the known processes of the prior art using fluidised-bed technology have the disadvantage that the polychlorinated dibenzo-p-dioxins/furans (PCDD/PCDF) formed in the reaction pass into the aqueous quench solution used for quenching. That quench solution has to be removed and sent for further working-up, which because of the PCDD/PCDF content of the quench solution is onerous and very expensive.

Moreover, the thermal energy of the hot process gases (=reaction gases) cannot be used in quenching.

A problem of the present invention is therefore to provide a process and an apparatus for the preparation of 1,2-dichloroethane, wherein the polychlorinated dibenzo-p-dioxins/furans formed during the reaction do not pass into an aqueous phase but remain in the organic phase.

It is a further problem of the present invention to provide a corresponding process and a corresponding apparatus wherein the thermal energy of the hot process gases can be used.

The problems are solved by provision of a process and an apparatus of the kind mentioned at the beginning, wherein the reaction gas formed during oxychlorination is condensed after filtration, without quenching. The process step of quenching, which has hitherto always been necessary in the art, is accordingly omitted from the fluidised-bed process. The process and the apparatus are therefore simpler and also, consequently, more economical.

In the case of such a process and such an apparatus, therefore, a so-called quenching tower does not need to be provided, which results in a saving of space and of investment costs.

Furthermore, in the case of the process according to the invention and the apparatus according to the invention, no PCDD/PCDFs pass into the aqueous phase so that the laborious and expensive working-up is omitted. Rather, the polychlorinated dibenzo-p-dioxins/furans (PCDD/PCDF) are separated out from the other components and then, for example, together with the other high-boiling components of the process, are sent for combustion.

As a result of the fact that, in the case of a process and the apparatus in accordance with the invention, no quenching is carried out, it is possible, according to a preferred embodiment of the present invention, for the thermal energy of the hot reaction gases to be used. Preferably, that energy serves for generating water vapour or for pre-heating the recycle gas/ethylene stream to the reactor, for example in a heat exchanger. The remainder of the heat (evaporation enthalpy of EDC and water) is preferably, in a further heat exchanger, transferred to a cooling medium such as cooling water. The vapour can be further used, for example, in an existing EDC/VC system (e.g. driving various product streams or heating distillation columns), resulting in an energy saving and also, consequently, in a reduction in costs.

A reactor customary per se can be used as the oxychlorination reactor. Fluidised-bed reactors, especially, have proved in practice to be advantageous for oxychlodnation. In the reaction there are formed reaction gases, which comprise mainly 1,2-dichloroethane, but also water, hydrogen chloride, PCDD/PCDF and catalyst fragments. They may further comprise unreacted ethene and chlorine.

Preferably, a catalyst is used for the oxychlorination step, CuCI₂ or FeCI₃ catalysts having been found to be especially suitable. CuCI₂ applied to a carrier has proved to be especially advantageous as catalyst. Suitable carriers are, for example, silicon dioxide, kieselguhr, fuller's earth, clay and aluminium oxide, with γ-aluminium oxide being preferred.

The process conditions, especially the oxychlorination step, can be performed preferably in accordance with the process conditions described in the German Auslegeschrift 1 518 931 and the German Patent Specification 1 468 489, the disclosures of which are hereby incorporated by reference in the present description.

As a result of the fact that the reaction gases are filtered after oxychlorination, more specifically preferably through a very fine filter, almost no catalyst contaminated with, for example, PCDD/PCDF passes into the aqueous phase but such catalyst remains in the filter. Very fine filtration is understood to be a procedure causing the fine portion of the oxychlorination catalyst to be retained. The fine portion has an average particle size of at least 1 μm. Filtration can be performed, for example, as described in the PCT Application PCT/EP98/07444. The disclosure of that publication is hereby incorporated by reference in the present description.

Provision may be made for the filtration to take place outside the oxychlorination reactor. That arrangement is especially advantageous when existing systems are to be retro-fifted.

In the case of new systems, it is, however, generally preferable for filtration to be carried out inside the oxychlorination reactor.

Filtration can be carried out in accordance with the invention by means of filter candles, bag filters and/or cartridge filters. Such filters are described, for example, in DE 197 53 165 A1 and are manufactured especially by Pall, Micropul, Fluiddynamics etc..

After filtering the reaction gas, cooling of the reaction gas—without quenching—is carried out, with, for example, pre-heating of the recycle gas passed to the reactor (ethylene mixture) and/or generation of water vapour which can be fed into the system vapour network and used for the heating of columns and pre-heaters. In a second heat exchanger, the reaction gas is partially condensed, and the heat is preferably transferred—for example in a heat exchanger—to a cooling medium, again without quenching. In a separator, the liquid phase is separated from the recycle gas and is sent for further working-up. That working-up is described in greater detail in DE 100 59 299.5, a copy of which is annexed. The EDC/water mixture, that is to say the organic and aqueous phase, is released into a container, whereupon the major part of the carbon dioxide is evolved from the EDC/water. The water is then sent for waste water treatment, the EDC is directed into an apparatus located downstream and the chloral and/or chloral hydrate contained therein is destroyed by treatment with an aqueous alkaline solution. In a decanter, the EDC is separated from the aqueous phase. The alkaline aqueous phase from the decanter is likewise sent for waste water treatment.

The EDC from the decanter is sent for distillation, for example in a so-called dehydration and low-boiler column and a high-boiler column. Such columns are known in EDC/VC systems. Low- and high-boilers, and corresponding components, are liquids having a boiling point lower and higher, respectively, than EDC. In the described arrangement of the invention, the polychlorinated dibenzo-p-dioxins/furans are removed in the high-boiler column together with the other high-boilers of the process and then sent, for example, to combustion.

In a further preferred embodiment of the present invention, introduction of at least one of the starting material streams, hydrogen chloride and oxygen-containing gas, is carried out by way of inlets having porous, gas-permeable packing elements. According to the invention, oxygen-containing gases may be, for example, air, oxygen and oxygen-containing gas mixtures. Introduction may be carried out directly into the fluidised bed of the oxychlorination reactor. Examples of such porous, gas-permeable packing elements are those manufactured by Pall, Fluid Dynamics, Krebsöge etc..

Introduction of the ethene and/or of the recycle gas into the oxychlorination reactor is carried out, in accordance with a further preferred embodiment, by way of a tray of porous, gas-permeable material. Examples of such porous, gas-permeable materials are VA-steel alloys, highly corrosion-resistant alloys, INCONEL®, MONEL®, HASTELLOY® and ceramic materials.

Preferably, both the oxygen-containing gas, on the one hand, and the ethene, on the other hand, are fed into the catalyst fluidised bed in finely distributed form, as described, for example, in DE 199 03 335 A1.

The inlets can be in the form described in DE 199 03 335 A1, which is hereby incorporated by reference in the description.

Preferably, the process according to the invention is carried out in an apparatus for the preparation of 1,2-dichloroethane by reacting ethene with hydrogen chloride and an oxygen-containing gas. That preferred apparatus has an oxychlorination reactor, a filter, a condenser and a 1,2-dichloroethane-distillation apparatus and is distinguished by the fact that there is furthermore provided a water vapour generator, but no quenching column.

The filter, which should be a very fine filter, can be composed of filter candles, bag filters and/or cartridge filters.

When filter candles are used, they should be made from materials suitable for EDC preparation. Such materials are, for example, metals, alloys, glass and/or ceramics. Preferably, the filter candles are of sintered metal and/or ceramics.

Furthermore, fabric filters made from sufficiently temperature-resistant, especially fluorinated, plastics materials such as polytetrafluoroethylene in the form of bag filters or cartridges could also be used.

It has, moreover, been found preferable to arrange the distillation apparatus so that it has a dehydration and low-boiler column and a high-boiler column.

In order to obtain products that are as pure as possible, the water vapour generator/starting material pre-heater of C steel and the condenser should, on their product side, be made of a nickel-containing material such as a nickel alloy, for example HASTELLOY® from Hayes International, Inc. or tantalum.

In addition, it would also be possible for the water vapour generator and the condenser, on their product side, to be made of graphite material, for example NS2 or NS3 from SIGRI.

Especially in order to be able to carry out a process in the manner described in greater detail hereinbefore, the apparatus should preferably have inlets for hydrogen chloride and oxygen-containing gas leading directly into the fluidised bed of the oxychlorination reactor.

Those inlets may comprise porous, gas-permeable packing elements.

It is also especially advantageous if the ethene and the recycle gas stream are passed into the oxychlorination reactor by way of a tray that is made from porous, gas-permeable material or that is provided with packing elements of porous, gas-permeable material.

Further advantages and developments of the invention are shown by the patent claims, the drawings, and the following description in which exemplary embodiments of the invention are described in detail with reference to the drawings.

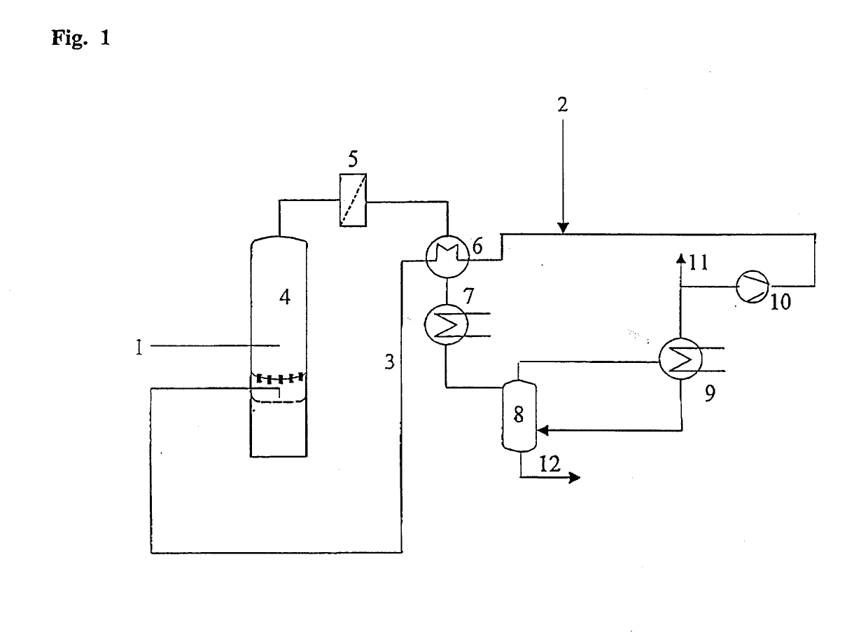

FIG. 1 shows an apparatus according to the invention for carrying out a process according to the invention in accordance with a first preferred embodiment of the present invention;

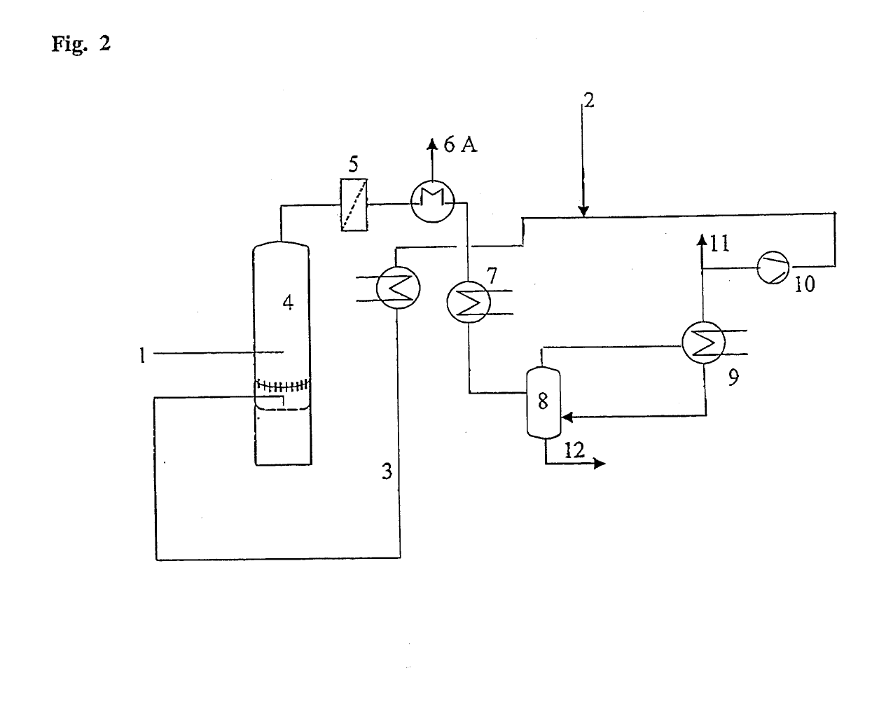

FIG. 2 shows an apparatus according to the invention for carrying out a process according to the invention in accordance with a second preferred embodiment of the present invention;

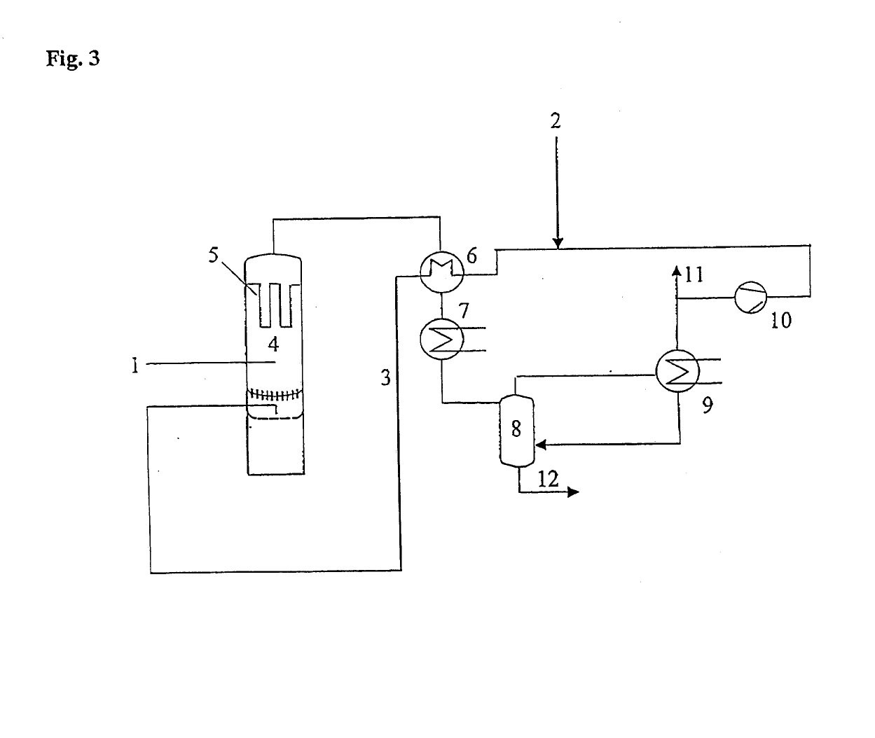

FIG. 3 shows an apparatus according to the invention for carrying out a process according to the invention in accordance with a third preferred embodiment of the present invention.

In FIG. 1 there is an apparatus for carrying out a process for the preparation of 1,2-dichloroethane by reacting ethene with hydrogen chloride and oxygen or an oxygen-containing gas in an oxychlorination reactor with formation of a reaction gas. Direct condensation with starting material pre-heating is described therein. The filter 5 is, in this instance, arranged outside the fluidised-bed reactor 4.

FIG. 1 shows a reactor 4, preferably a fluidised-bed reactor, into which there lead two lines 1 and 3, through which the process gases are introduced. Hydrogen chloride and oxygen are fed into the reactor 4 by way of line 1 and ethylene and recycle gas by way of line 3. Line 3 has a heat exchanger 6, in which the waste heat of the reaction gases emerging from the reactor is used for pre-heating the ethylene (or, also, the gas referred to as “ethene”) and/or the recycle gas. Ethylene is fed into the system by way of the inlet line 2. Downstream of the reactor 4 is the filter 5, by means of which the hot reaction gases emerging from the reactor are freed from solid constituents. The reaction gases are cooled in the heat exchanger 6, before they are introduced into the condenser 7, the waste heat of which can also be utilised by means of a heat exchanger.

On leaving the condenser 7, the reaction gas still has a temperature of about 60° C. At that temperature, the mixture, which comprises a 1,2-dichloroethane-containing organic phase and an aqueous phase, is introduced into the separating apparatus 8.

From that separating apparatus 8, there is separated out the product-containing liquid mixture, comprising an organic phase and an aqueous phase, by way of line 12, from the gaseous phase, which is used further as recycle gas by way of the heat exchanger 9 and the recycle gas compressor 10. Upstream of the recycle gas compressor 10 there is provided a waste gas line 11.

In accordance with the preferred embodiment shown in FIG. 1, the reaction gas is filtered after emerging from the fluidised-bed reactor and is then condensed without prior quenching.

FIG. 2 then shows a further preferred embodiment of the present invention, the same reference symbols being used for components that correspond to FIG. 1.

FIG. 2 shows a comparable schematic circuit diagram of a system wherein, instead of or in addition to the process gas pre-heating in the heat exchanger 6, the waste heat of the reaction gases is used by means of water vapour generation in the heat exchanger 6A.

Finally, FIG. 3 shows a reactor 4 having an internally located filter 5 so that filtration of the hot reaction gases takes place whilst they are still in the reactor 4 and, as a result of the filtration, minimal heat is lost before introduction of the reaction gases into a heat exchanger 6, which is used for pre-heating the process gases and/or for vapour generation, especially water vapour generation. The rest of the system is unchanged with respect to that shown in FIGS. 1 and 2.

### EXAMPLE

An oxychlorination reactor having a fluidised bed is used for the preparation of 1,2-dichloroethane, with CuCI₂ being used as catalyst. The oxychlorination is carried out as follows:

5910 Nm³/h of hydrogen chloride at a temperature of 150° C. and 1600 Nm³/h of oxygen, heated to 110° C., are introduced, by way of line 1, directly into the fluidised bed (40 t of catalyst; aluminium oxide having a copper content of 4% by weight) of the reactor 4 by way of inlets 1, 3, which comprise porous, gas-permeable packing elements, for example of sintered chromium-nickel-steel from Pall.

Introduction of the ethene (3000 Nm³/h) and of the recycle gas stream is carried out by way of a tray in the oxychlorination reactor 4, the tray being made from porous, gas-permeable material. After oxychlorination and after leaving the fluidised bed, the hot (200-250° C.) reaction gas, consisting of EDC, H₂O, CO₂, CO, nitrogen, C₂H₄, HCI and O₂, for the purpose of separating out entrained catalyst particles, in this instance CuCI₂, flows through a very fine filter 5 in the upper region of the oxychlorination reactor 4, in which filter the catalyst is separated out.

The hot (about 200 to 250° C.) reactor head gas is cooled to about 140° C. in a suitable apparatus, a tube bundle heat exchanger made from C steel. The energy given off therein is used to produce water vapour. The water vapour is fed into the water vapour network of the system and is further used for distillation of EDC in the high-boiler or low-boiler columns.

The vapour produced in that manner at a temperature of 135° C. and a pressure of 3 bar is used in the distillation column of the existing EDC/VC system (high-boiler or low-boiler column).

In a further cooling phase, the reactor head gas is further cooled to about 60° C. using a tube bundle heat exchanger made from an acid-resistant material, for example from NS1 graphite from Sigri, and, in the process, the produced EDC and water are condensed from the recycle gas stream. The energy given off is transferred to the cooling water.

The water vapour generator 6A is a horizontally arranged tube bundle heat exchanger, in which the OC process gas is passed through the tubes and the water vapour is delivered into an enlarged shell space by way of a pressure-retaining valve into the vapour network of the system.

The condenser 7 is, on the product side, made from WS2 graphite from Sigri, so that no further contamination of the reaction gas occurs. The condensed product stream is then sent for EDC distillation, wherein the PCDD/PCDFs are separated out, as a result of distillation, together with the so-called high-boilers and are subsequently combusted.

The PCDD/PCDFs contained in the crude EDC gas are removed, by way of the sump of the low-boiler column, to the high-boiler column. In the high-boiler column, those boiling-resistant components are removed, by way of the sump, to the vacuum column, from where they are combusted, together with the high-boiler residues, in thermal residue combustion at 1200° C.

1. Process for the preparation of 1,2-dichloroethane by reacting ethene with hydrogen chloride and oxygen or an oxygen-containing gas with formation of a reaction gas, characterised in that the reaction gas is filtered and is then condensed without quenching.

2. Process according to claim 1, characterised in that thermal energy of the reaction gas is used especially for the generation of vapour and/or thermal energy is used for cross-heat-exchanging (heating up) of various product streams (preferably recycle gas).

3. Process according to claim 1 or 2, characterised in that the oxychlorination reactor comprises a fluidised-bed reactor (4).

4. Process according to one of claims 1 to 3, characterised in that the filtration is carried out inside the oxychlorination reactor.

5. Process according to one of claims 1 to 3, characterised in that the filtration is carried out outside the oxychlorination reactor.

6. Process according to one of the preceding claims, characterised in that the filtration is carried out by means of filter candles, bag filters and/or cartridge filters.

7. Process according to one of the preceding claims, characterised in that, after the condensation, the 1,2-dichloroethane-containing phase is decanted off from the aqueous phase and is sent for 1,2-dichloroethane distillation.

8. Process according to claim 7, characterised in that the 1,2-dichloroethane distillation is carried out first in a dehydration and low-boiler column and then in a high-boiler column.

9. Process according to claim 8, characterised in that polychlorinated dibenzo-p-dioxins/furans are separated out in the high-boiler/vacuum column.

10. Process according to one of the preceding claims, characterised in that hydrogen chloride, ethene, recycle gas and/or the oxygen-containing gas are introduced into the oxychlorination reactor through porous, gas-permeable packing elements.

11. Apparatus for the preparation of 1,2-dichloroethane by reacting ethene with hydrogen chloride and an oxygen-containing gas, especially for use in a process according to one of the preceding claims, having an oxychlorination reactor, a filter, a condenser and a 1,2-dichloroethane-distillation apparatus, characterised in that there are further provided a water vapour generator (6A)/or starting material pre-heater (6) and a condenser (7) operated with cooling water, and no quenching column.

12. Apparatus according to claim 11, characterised in that the filter (5) comprises filter candles, a bag and/or cartridge filters.

13. Apparatus according to claim 12, characterised in that the filter candles are of sintered metal and/or ceramics.

14. Apparatus according to one of claims 11 to 13, characterised in that the distillation apparatus has a dehydration and low-boiler column and a high-boiler column.

15. Apparatus according to one of claims 11 to 14, characterised in that the condenser (7) is, on its product side, made of a nickel-containing material such as a nickel alloy.

16. Apparatus according to one of claims 11 to 15, characterised in that the condenser (7) is, on its product side, made of a graphite material.

17. Apparatus according to one of claims 11 to 16, characterised in that the oxychlorination reactor is a fluidised-bed reactor (4).

18. Apparatus according to claim 17, characterised in that the inlets (1) for hydrogen chloride and oxygen-containing gas lead directly into a fluidised bed of the fluidised-bed reactor (4).

19. Apparatus according to claim 18, characterised in that the inlets (1) comprise porous, gas-permeable packing elements.

20. Apparatus according to one of claims 11 to 19, characterised in that the recycle gas and/or the ethene is/are passed into the oxychlorination reactor through a tray of porous, gas-permeable material.
